(19) United States
(12) Patent Application Publication
(10) Pub. No.: US 2025/0143925 A1
(43) Pub. Date: May 8, 2025

(54) SURGICAL INSTRUMENTS HAVING REDUCED ADHERENCE TO VITREOUS

(71) Applicant: Alcon Inc., Fribourg (CH)

(72) Inventors: Niels Alexander Abt, Winterthur (CH); Michael Sam Cardamone, Keller, TX (US); Reto Grüebler, Greifensee (CH); Paul R. Hallen, Colleyville, TX (US)

(21) Appl. No.: 18/898,967

(22) Filed: Sep. 27, 2024

Related U.S. Application Data
(60) Provisional application No. 63/597,092, filed on Nov. 8, 2023.

(51) Int. Cl. A61F 9/007

(52) U.S. Cl. CPC A61F 9/00745

(57) ABSTRACT

A surgical instrument for ophthalmic surgery includes a handle comprising a distal end and a probe comprising a proximal end coupled to the distal end of the handle. In some embodiments, a micro-structure pattern or a coating is formed on a surface of the probe. The surgical instrument further includes a driver in communication with the probe that is configured to cause vibration of the probe. The micro-structure pattern or coating and the vibration may be configured to reduce adhesion of the probe to a vitreous of an eye.

### BACKGROUND

Anatomically, the human eye is divided into two distinct regions: the anterior segment and the posterior segment. The anterior segment includes the lens and extends from the outermost layer of the cornea to the posterior of the lens capsule. The posterior segment of the eye includes the anterior hyaloid membrane and all of the ocular structures behind it, such as the vitreous humor, retina, choroid, and the optic nerve.

Vitreoretinal surgery is performed within the posterior segment of the human eye to treat serious conditions including, but not limited to, age-related macular degeneration (AMD), diabetic retinopathy, diabetic vitreous hemorrhage, macular holes, retinal detachment, epiretinal membrane, diabetic retinopathy, and cytomegalovirus retinitis. Such procedures frequently require the severance and removal of portions of the vitreous humor from the posterior segment of the eye, which is a colorless, gel-like substance made of water, collagen and hyaluronic acid.

Vitreoretinal surgery may require incisions and insertion of surgical instruments within an eye to remove the vitreous and/or repair the retina. The vitreous and the retina are connected via small microscopic fibrils of vitreous that are attached to the retina and, therefore, removal of the vitreous must be done with great care to avoid disturbing the retina. If the vitreous is disturbed during the surgery or incorrectly/inadvertently manipulated, the retina may be subject to unwanted traction causing separation of the retina from the choroid in the back of the eye, a retinal tear, or removal of the retina itself with the vitreous.

There have been improvements in the art, such as increased vitreous cutting speeds, that have improved the amount of traction produced in vitreoretinal surgery; however, the incidence rate of retinal tears is still high. Therefore, improved instruments and techniques for preventing retinal tears and other retinal damage during vitreoretinal surgery are desirable.

### SUMMARY

Aspects of the present disclosure relate to instruments for ophthalmic (eye) procedures, and more specifically, surgical instruments for vitreoretinal surgical procedures.

In certain embodiments, a surgical instrument for ophthalmic procedures is provided. The surgical instrument includes a handle comprising a distal end and a probe comprising a proximal end coupled to the distal end of the handle and a micro-structure patterned on an interior or exterior (or both) surface of the probe.

In certain embodiments, a surgical instrument includes a handle comprising a distal end, and a probe comprising a proximal end coupled to the distal end of the handle and a driver in communication with the probe. The driver is configured to cause vibration of the probe.

In certain other embodiments, a surgical instrument includes a handle comprising a distal end, and a probe comprising a proximal end coupled to the distal end of the handle and a coating on the distal end of the probe. The coating may be on the interior or exterior (or both) surface of the distal end of the probe. The coating is configured to reduce adhesion to a vitreous of an eye.

### BRIEF DESCRIPTION OF THE DRAWINGS

The drawings described herein are for illustrative purposes only, are schematic in nature, and are intended to be exemplary rather than to limit the scope of the disclosure.

FIG. 1 illustrates a perspective view of an exemplary surgical instrument, according to embodiments described herein.

FIGS. 2A-2C illustrate perspective views of exemplary surgical instruments having vibrating drivers, according to embodiments described herein.

FIG. 3 illustrates a detailed perspective view of an exemplary probe of a surgical instrument comprising a micro-structure on the exterior surface of the probe, according to embodiments described herein.

FIG. 4 illustrates a detailed cross-sectional view of an exemplary probe of a surgical instrument comprising a coating on the exterior surface of the probe, according to embodiments described herein.

FIG. 5 illustrates a detailed cross-sectional view of an exemplary probe of a surgical instrument comprising a coating on the interior surface of the probe, according to embodiments described herein.

FIG. 6 illustrates a detailed cross-sectional view of an exemplary probe of a surgical instrument comprising a coating on the interior and exterior surface of the probe, according to embodiments described herein.

The above summary is not intended to represent every possible embodiment or every aspect of the subject disclosure. Rather the foregoing summary is intended to exemplify some of the novel aspects and features disclosed herein. The above features and advantages, and other features and advantages of the subject disclosure, will be readily apparent from the following detailed description of representative embodiments and modes for carrying out the subject disclosure when taken in connection with the accompanying drawings and the appended claims.

### DETAILED DESCRIPTION

Aspects of the present disclosure relate to ophthalmic procedures, and more particularly, to instruments for use during ophthalmic procedures.

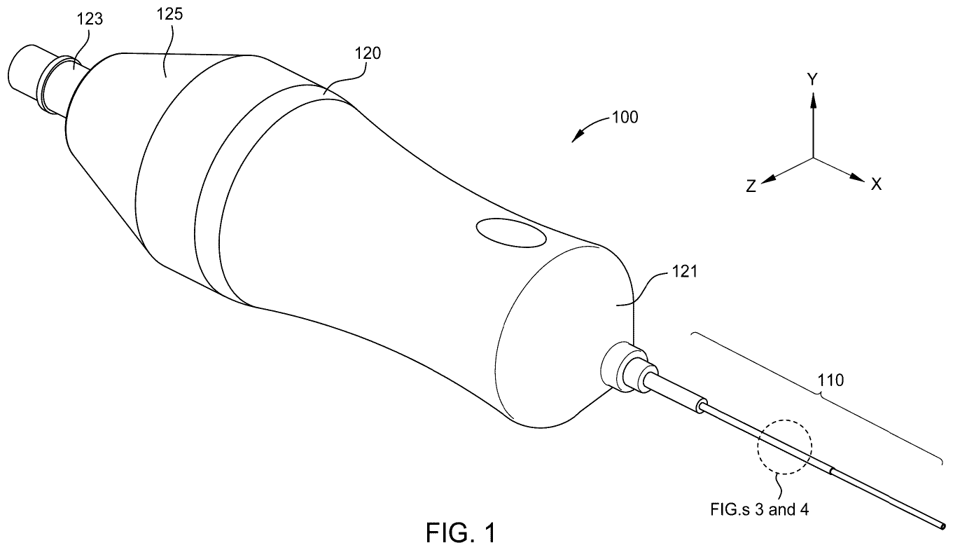

FIG. 1 illustrates a perspective view of an exemplary surgical instrument according to embodiments described herein. As depicted in FIG. 1, the surgical instrument 100 comprises a probe 110 and a base 120. The probe 110 is partially and longitudinally disposed through a distal end 121 of the base 120 and may be directly or indirectly attached thereto within an interior chamber of the base 120. Note that, as described herein, a distal end or portion of a component refers to the end or the portion that is closer to the patient's body during the use thereof. On the other hand, a proximal end or portion of the component refers to the end or portion that is distanced further away from the patient's body.

In some embodiments, the base 120 is a hand piece having an outer surface configured to be held by a user, such as a surgeon. For example, the base 120 may be ergonomically contoured to substantially fit the hand of the user. In some embodiments, the outer surface may be textured and/or have one or more gripping features formed thereon, such as one or more grooves and/or ridges. The base 120 may be made from any materials commonly used for such instruments and suitable for ophthalmic surgery. For example, the base 120 may be formed of a lightweight aluminum, a polymer, or other suitable material. In some embodiments, the base 120 may be sterilized and used in more than one surgical procedure, or it may be a single-use device.

The base 120 further provides one or more ports 123 (e.g., one port is depicted in FIG. 1) at a proximal end 125 thereof for one or more supply lines to be routed into an interior chamber of the base 120. For example, the port 123 may provide a connection between the base 120 and a vacuum line of a vacuum source for aspiration. The port 123 may also provide a connection to an optical fiber cable that couples to one or more light sources for providing laser light as well as illumination light. In the same or other embodiments, the base 120 may include a driver to connect to a cutting tip at the distal end of the probe 110. The cutting tip may be vibrated ultrasonically (e.g., in a torsional manner) to sever vitreous fibrils without laser light, for example.

The probe 110 extends from the distal end of the base unit 120 and may have an outer surface configured to interface with the external environment (e.g., vitreous within a patient's eye). The probe 110 may be made of any materials commonly used for such instruments and suitable for ophthalmic surgery. For example, the probe 110 may be formed of a lightweight aluminum, a polymer, or other suitable material. In some embodiments, the probe 110 may be sterilized and used in more than one surgical procedure, or it may be a single-use device.

While the embodiments described herein generally refer to a surgical instrument as shown in FIG. 1, the following disclosure may relate to any surgical instrument, including, but not limited to a vitrectomy probe, a cannula, and forceps.

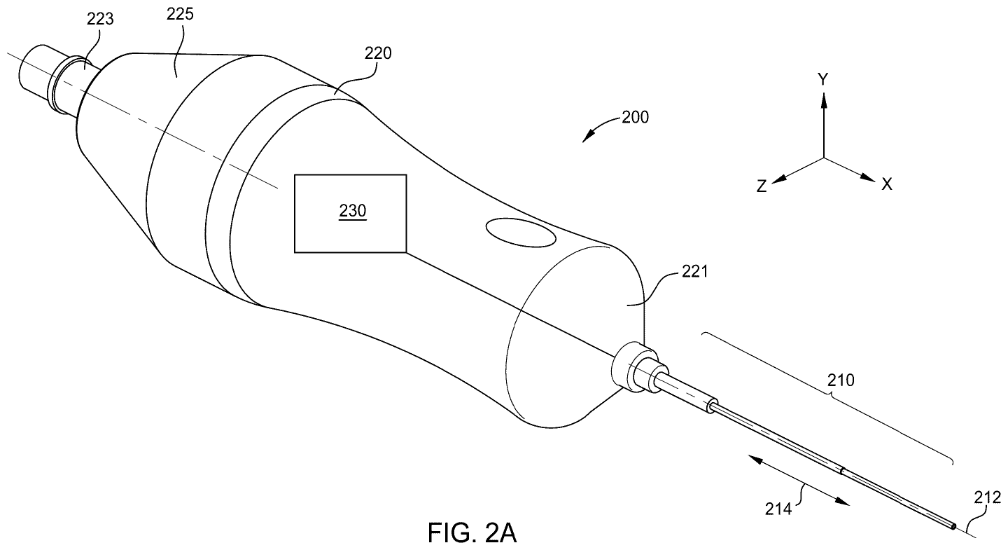

FIGS. 2A-2C illustrate perspective views of exemplary surgical devices having vibrating drivers, according to embodiments described herein. As shown, the surgical instrument 200 generally includes a base 220 and a probe 210 coupled to and extending distally from the distal end 221 of the base 220. In certain embodiments, the base 220 includes a vibration driver (e.g., vibration driver 230, 250, or 270) which may be coupled to the probe 210 via connection 222 to actuate vibration of the probe 210 (e.g., in rotational vibration, a gyrational vibration, or an axial vibration).

Vibration of the probe 210 prevents vitreous fibrils in the eye from adhering to the probe 210 as the probe 210 enters a patient's eye. In order to prevent adhesion of vitreous fibrils, the surgical instrument 200 may vibrate (e.g., oscillate, pulsate, etc.) at a high-frequency, low-amplitude vibration. For example, the surgical instrument 200 may vibrate at a frequency between 1 kHz (kilohertz)-1 MHz (megahertz), such as between 1-500 kHz, or between 1-250 kHz, or between 1-100 kHz, or between 1-100 MHz, or between 1-50 MHz, or between 50-100 MHz. Further, the surgical instrument 200 may vibrate at an amplitude between 1-500 micrometers, such as between 1-250 micrometers, or between 1-100 micrometers, or between 1-50 micrometers, or between 100-200 micrometers, or between 200-500 micrometers, for example. The high-frequency, low-amplitude vibration may be preferred as low-amplitude vibration causes little displacement within the eye, while high-frequency vibration provides sufficient velocity to overcome the friction of the vitreous with the surgical instrument 200. As described herein, reduction in adhesion of the vitreous may prevent retinal tears and other retinal damage that results from ophthalmic procedures, specifically vitreoretinal surgery.

In FIG. 2A, the surgical instrument 200 is shown with a surgical instrument axis 212, (also referred to herein as the longitudinal axis of the surgical instrument) which is also the major axis of the probe 210. The vibration driver 230 housed in the base 220 may be coupled to the probe 210 via connection 222 to cause the probe 210 to be vibrated in an axial manner (e.g., parallel to the surgical instrument axis 212). The pattern of vibration is demonstrated by arrows 214.

In FIG. 2B, the surgical instrument 200 is shown with a surgical instrument axis 212, similar to FIG. 2A. The vibration driver 250 housed in the base 220 may be connected to the probe 210 via connection 222. The vibration driver 250 may cause the probe 210 to vibrate in a rotational manner. Rotational vibration may refer to circular movement of the probe 210 about the surgical instrument axis 212, while the axis of the probe 210 remains aligned with the surgical instrument axis. The circular movement of the probe 210 may be continuous movement in a single direction (e.g., clockwise or counterclockwise) or movement in a back-and-forth manner (e.g., cyclical). The pattern of vibration is demonstrated by arrows 216.

In FIG. 2C, the surgical instrument 200 shown has a surgical instrument axis 212, similar to FIG. 2A. The vibration driver 270 housed in the base 220 may vibrate the surgical instrument 200 and/or the probe 210 in a gyrational manner. In some embodiments, gyrational vibration may refer to a movement of the probe 210 in a circular or oval pattern relative to the surgical instrument axis 212.

For example, as shown in FIG. 2C, the probe 210 may vibrate a distance away from the surgical instrument axis 212 in a circular motion about the axis. While the distal end of the probe 210 may rotate a greater distance from the surgical instrument axis 212, a majority of the length of the probe 210 may rotate a distance from the surgical instrument axis 212. In other words, the rotation of the probe may be a conical path such that the distal end of the probe 210 rotates the greatest distance from the surgical instrument axis 212 and the proximal end of the probe 210 rotates at the surgical instrument axis 212. The pattern of vibration is demonstrated by arrows 218 and further illustrated by the “phantom” probes depicted below and above the probe 210. While the “phantom” probes in FIG. 2C demonstrate a visible rotation path for illustrative purposes, the present disclosure also contemplates a small-scale rotation (e.g., not visible to the naked eye).

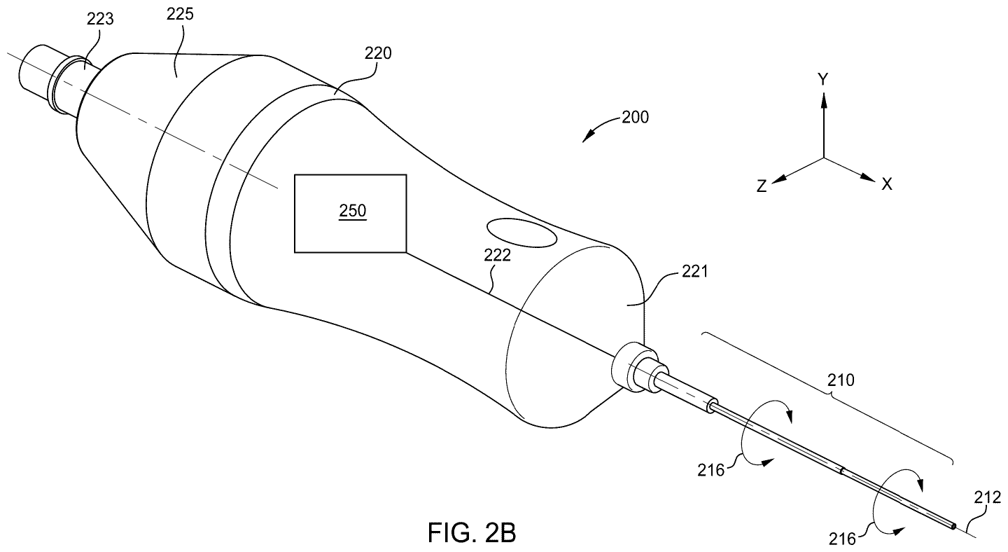

FIG. 3 illustrates a detailed perspective view of an exemplary probe 310 of a surgical instrument 300 comprising a micro-structure 302 on the exterior surface of the probe 310. The surgical instrument may be surgical instrument 200 comprising a vibration driver (e.g., vibration driver 230, 250, or 270), or may be another surgical instrument not comprising a vibration driver.

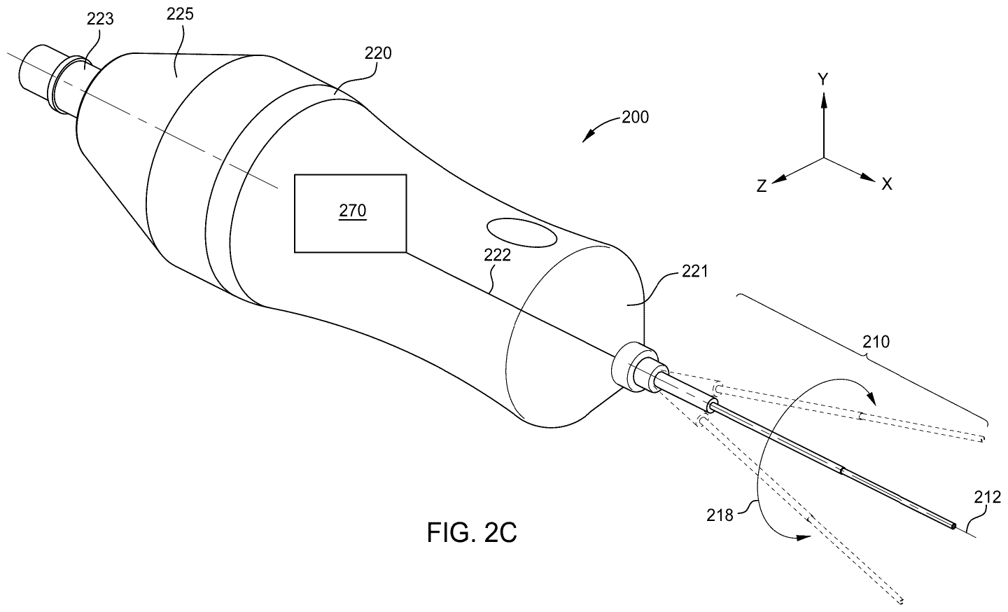

As shown, the micro-structure 302 includes raised surfaces on the exterior surface of the probe 310, which may cover the entire length of the probe 310 or just a portion of the probe 310 (e.g., a portion of the probe that enters the eye of a patient). In some embodiments, the micro-structure 302 includes raised surfaces on the interior surface of the probe 310, which may cover the entire length of the probe 310 or just a portion of the probe 310 (e.g., a portion of the probe that enters the eye of a patient). As discussed relative to the probe in FIG. 1, the probe 310 may be made of any materials, such as materials that are commonly used for such instruments and suitable for ophthalmic surgery. For example, the probe 310 may be formed of a lightweight aluminum, a polymer, or other suitable material. Based on the material of the probe 310, the micro-structure 302 may be applied to the interior or exterior surface of the probe 310 using any suitable method, including, without limitation, chemical etching, physical etching (e.g., laser etching), wet etching, dry etching, stamping, or molding. As discussed herein in reference to FIG. 4, the micro-structure 302 may also be applied to the probe 310 via a coating.

The micro-structure 302 may be operable to create a non-adherent or low adherence interface between the probe 310 and the vitreous of the eye of the patient during surgery. In certain embodiments, the non-adherent interface may be hydrophobic. As shown, the micro-structure 302 comprises a diamond pattern of raised surfaces on the probe 310. However, the pattern may be any suitable pattern comprising raised surface area on the probe 310, such as a pattern of small “pillars” raised from the surface of the probe 310 in any suitable pattern, for example. The micro-structure may have a depth of 0.1 microns to 100 microns from the surface of the probe 310, such as a depth between 0.1 microns and 100 microns, such as between 0.1 microns and 50 microns, such as between 1 micron and 50 microns, such as between 1 micron and 10 microns.

In certain embodiments, the micro-structure 302 may be a nano-crystalline diamond material. The nano-crystalline diamond material may be created by growing millions of small diamond crystals to be placed on the surface of the probe 310. A nano-crystalline diamond material on the probe 310 may create a raised surface area on the interior or exterior surface of the probe 310 and create a low friction interface between the probe 310 and the vitreous fibrils.

In certain other embodiments, the micro-structure 302 may describe a polished or roughened surface of the probe 310. For example, the interior or exterior surface of the probe 310 may be polished using a physical polishing technique in order to lessen friction between the interior or exterior surface of the probe 310 and the eye of the patient (e.g., vitreous fibrils).

FIG. 4 illustrates a detailed cross-sectional perspective view of an exemplary probe 410 of a surgical instrument comprising a coating on the exterior surface of the probe 410. The surgical instrument may be surgical instrument 200 comprising a vibration driver (e.g., vibration driver 230, 250, or 270), or may be another surgical instrument not comprising a vibration driver.

As shown, coating 404 may be a permanent or semi-permanent coating disposed on the exterior surface of the probe 410 to create a low-adherence or non-adherent interface with a vitreous of an eye of a patient. The coating may be configured to reduce adhesion to the vitreous of the eye of the patient. The reduction in adherence of the coating may be measured when compared to the adhesion of the material of the probe (e.g., lightweight aluminum, a polymer, or other suitable material) to the vitreous of the eye of the patient. In certain embodiments, the coating 404 may be hydrophobic. The coating 404 on the exterior surface of the probe 410 may be made of any material suitable to create a low-adherence, non-adherent, or hydrophobic interface with an eye of a patient. For example, the coating may be formed of polyvinyl chloride, polyurethane, polytetrafluoroethylene, epoxy resin, silicon, polysilicon (e.g., formed via a plasma deposition process), hydrophilic amine, sulfonate, multilayer polyelectrolyte, hydroxyl, amine (e.g., a polymer substrate having an amine incorporated into the polymer substrate), sulfonate, multilayer polyelectrolyte, polydimethyl siloxane, phospholipid bilayer, hyaluronic acid-based materials, carbon nanotubes, polytetrafluoroethylene, physical vapor deposition (PVD)-formed materials, ceramics, or other suitable materials. The coating 404 may have a thickness of 0.01 microns to 100 microns along the length of the probe 410.

In certain embodiments, the coating 404 may be applied to the probe 410 to create a micro-structure (e.g., micro-structure 302). For example, the coating 404 may create a micro-structure on the exterior surface of the probe 410. The micro-structure created by the coating 404 may be any suitable pattern to prevent adhesion of the vitreous to the probe 410.

In certain embodiments, the coating 404 may be a temporary coating. For example, the temporary coating may be formed of silicone oil, sodium chloride, perfluorocarbon liquid, or other suitable material. The temporary coating may be applied to the surgical instrument (e.g., surgical instrument 200 or surgical instrument 300) by the surgeon by dipping the probe of the surgical instrument into the temporary coating. Immediately following application of the temporary coating, the surgeon may then insert the surgical instrument directly into the eye of the patient.

In certain embodiments, the coating may be a coating that prevents adhesion by osmosis (e.g., an osmotic coating). In still other embodiments, the coating 404 may be a dry coating (e.g., a non-liquid material) applied to the probe to create the low-adherence, non-adherent, or hydrophobic interface with an eye of a patient.

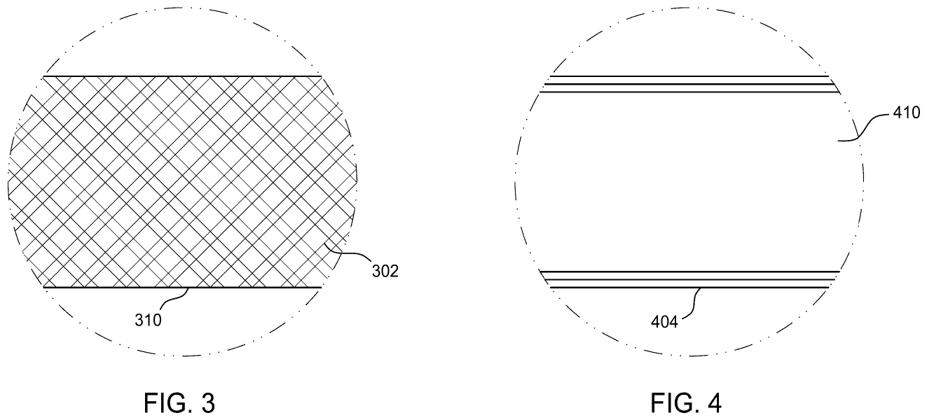

FIG. 5 illustrates a detailed cross-sectional perspective view of an exemplary probe 510 of a surgical instrument comprising a coating on the interior surface of the probe 510. The surgical instrument may be surgical instrument 200 comprising a vibration driver (e.g., vibration driver 230, 250, or 270), or may be another surgical instrument not comprising a vibration driver.

As shown, coating 504 may be a permanent or semi-permanent coating disposed on the interior surface of the probe 510 (e.g., on the interior surface of wall 512) to create a low-adherence or non-adherent interface with a vitreous of an eye of a patient. The coating may be configured to reduce adhesion to the vitreous of the eye of the patient. The reduction in adherence of the coating may be measured when compared to the adhesion of the material of the probe (e.g., lightweight aluminum, a polymer, or other suitable material) to the vitreous of the eye of the patient. In certain embodiments, the coating 504 may be hydrophobic. The coating 504 on the interior surface of the probe 510 may be made of any material suitable to create a low-adherence, non-adherent, or hydrophobic interface with an eye of a patient. For example, the coating may be formed of polyvinyl chloride, polyurethane, polytetrafluoroethylene, epoxy resin, silicon, polysilicon (e.g., formed via a plasma deposition process), hydrophilic amine, sulfonate, multilayer polyelectrolyte, hydroxyl, amine (e.g., a polymer substrate having an amine incorporated into the polymer substrate), sulfonate, multilayer polyelectrolyte, polydimethyl siloxane, phospholipid bilayer, hyaluronic acid-based materials, carbon nanotubes, polytetrafluoroethylene, physical vapor deposition (PVD)-formed materials, ceramics, or other suitable materials. The coating 504 may have a thickness of 0.01 microns to 100 microns along the length of the probe 510.

In certain embodiments, the coating 504 may be applied to the probe 510 to create a micro-structure (e.g., micro-structure 302). For example, the coating 504 may create a micro-structure on the interior surface of the probe 510. The micro-structure created by the coating 504 may be any suitable pattern to prevent adhesion of the vitreous to the probe 510.

In certain embodiments, the coating 504 may be a temporary coating. For example, the temporary coating may be formed of silicone oil, sodium chloride, perfluorocarbon liquid, or other suitable material. The temporary coating may be applied to the surgical instrument (e.g., surgical instrument 200 or surgical instrument 300) by the surgeon by dipping the probe of the surgical instrument into the temporary coating. Immediately following application of the temporary coating, the surgeon may then insert the surgical instrument directly into the eye of the patient.

In certain embodiments, the coating may be a coating that prevents adhesion by osmosis (e.g., an osmotic coating). In still other embodiments, the coating 504 may be a dry coating (e.g., a non-liquid material) applied to the probe to create the low-adherence, non-adherent, or hydrophobic interface with an eye of a patient.

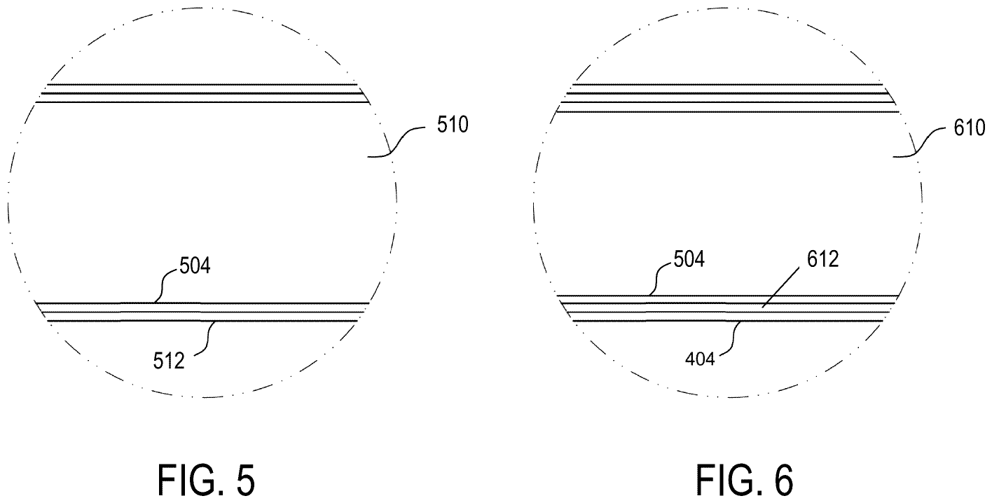

FIG. 6 illustrates a detailed cross-sectional perspective view of an exemplary probe 610 of a surgical instrument comprising a coating on the interior surface and the exterior surface of the probe 610. The surgical instrument may be surgical instrument 200 comprising a vibration driver (e.g., vibration driver 230, 250, or 270), or may be another surgical instrument not comprising a vibration driver.

As shown, coating 504 may be a permanent or semi-permanent coating disposed on the interior surface of the probe 610 (e.g., on the interior surface of wall 612) to create a low-adherence or non-adherent interface with a vitreous of an eye of a patient and coating 404 may be a permanent or semi-permanent coating disposed on the exterior surface of the probe 610 (e.g., on the exterior surface of wall 612) to create a low-adherence or non-adherent interface with a vitreous of an eye of a patient. The coating(s) 404/504 may be configured to reduce adhesion to the vitreous of the eye of the patient. In some embodiments, coatings 404 and 504 may have the same material/configuration. In some embodiments, coatings 404 and 504 may be different (e.g., coating 404 may be made of polytetrafluoroethylene while coating 504 may be a ceramic. Other coating choices are also contemplated. In some embodiments, one of the coatings 404 or 504 may be applied (to an interior or exterior surface, respectively) and the other of the interior or exterior surface may have a micro-structure applied). Other combinations of coatings and micro-structures are also contemplated.

The above disclosed subject matter is to be considered illustrative, and not restrictive, and the appended claims are intended to cover all such modifications, enhancements, and other embodiments which fall within the true spirit and scope of the present disclosure. Thus, to the maximum extent allowed by law, the scope of the present disclosure is to be determined by the broadest permissible interpretation of the following claims and their equivalents, and shall not be restricted or limited by the foregoing detailed description.

What is claimed is:

1. A surgical instrument, comprising:
a handle comprising a distal end;
a probe comprising:
a proximal end coupled to the distal end of the handle;
a micro-structure pattern or a coating formed on a surface of the probe;
a driver in communication with the probe, wherein the driver is configured to cause vibration of the probe; and
wherein the micro-structure pattern or coating and the vibration are configured to reduce adhesion of the probe to a vitreous of an eye.

2. The surgical instrument of claim 1, wherein the micro-structure is patterned on an interior or an exterior surface of a probe.

3. The surgical instrument of claim 1, wherein the coating is formed on an interior or an exterior surface of a probe.

4. The surgical instrument of claim 1, wherein the micro-structure is patterned on an exterior of the probe and the coating is formed on an interior of the probe.

5. The surgical instrument of claim 1, wherein the micro-structure is patterned on an interior of the probe and the coating is formed on an exterior of the probe.

6. The surgical instrument of claim 1, wherein the micro-structure on the surface of the probe is formed by chemical etching, physical etching, wet etching, dry etching, stamping, or molding.

7. The surgical instrument of claim 1, wherein a depth of the micro-structure is between 0.1 microns and 100 microns.

8. The surgical instrument of claim 1, wherein the driver is configured to cause at least one of a rotational vibration, a gyrational vibration, or an axial vibration of the probe.

9. The surgical instrument of claim 8, wherein the vibration is at a frequency between 1-100 MHz (Megahertz) and an amplitude between 1-500 micrometers.

10. The surgical instrument of claim 8, wherein the vibration comprises a rotational vibration comprising a continuous circular movement of the probe about a longitudinal axis of the surgical instrument.

11. The surgical instrument of claim 8, wherein the vibration comprises a gyrational vibration comprising a rotational movement of the probe in a conical path about a longitudinal axis of the surgical instrument.

12. The ophthalmic surgical instrument of claim 1, wherein the coating on the probe comprises a hydrophobic material having a thickness of 0.01 microns to 100 microns.

13. The ophthalmic surgical instrument of claim 1, wherein the coating forms a micro-structure on the distal end of the probe.

14. The ophthalmic surgical instrument of claim 1, wherein the coating forms a hydrophobic interface on a distal end of the probe.

15. The ophthalmic surgical instrument of claim 1, wherein the coating comprises a ceramic or polytetrafluoroethylene.
